(19) United States
(12) Patent Application Publication
(10) Pub. No.: US 2021/0315849 A1
(43) Pub. Date: Oct. 14, 2021

(54) COMPOSITIONS AND METHODS FOR TREATING ONE OR MORE AILMENTS WITH A NASAL RINSE COMPRISING N-ACETYLCYSTEINE

(71) Applicant: Pacific Sinus Solutions LLC, Henderson, NV (US)

(72) Inventors: Brian Weeks, San Diego, CA (US); Ashley Sikand, Las Vegas, NV (US); Raymond J. Faltinsky, Rancho Santa Fe, CA (US); Sean Ross, San Diego, CA (US)

(21) Appl. No.: 16/846,138

(22) Filed: Apr. 10, 2020

(51) Int. Cl. A61K 31/198; A61K 9/00

(52) U.S. Cl. CPC A61K 31/198; A61K 9/14; A61K 9/0043

(57) ABSTRACT

Compositions and methods for introducing a nasal rinse into one or more nasal passages to treat one or more potential ailments are disclosed. The compositions include N-acetylcysteine, a salt, and an alkaline buffering agent. The methods include introducing the composition into an aqueous fluid to form a nasal rinse treatment fluid and introducing the nasal rinse treatment fluid into one or more nasal passages to treat one or more potential ailments.

### BACKGROUND

The present disclosure relates generally to compositions and methods for introducing a nasal rinse into one or more nasal passages to treat one or more potential ailments, and more particularly, to such compositions and methods including N-acetylcysteine.

A nasal passage runs from the nostrils to the pharynx and is also lined with a delicate membrane called mucosa. Inflammatory disease of a nasal passage encompasses disorders that include acute and chronic sinusitis, allergic and non-allergic rhinitis, and viral and bacterial nasopharyngitis. A unifying pathogenesis for these conditions is an inflammatory response that is incited by antigens or variable pathogens, including bacteria and viruses. This inflammation is ultimately modulated by the immunologic system, which results in development of nasal congestion, mucus drainage, and impairment of sinus function that instigates facial pressure and pain. For the foregoing reasons, there is a need for a treatment to decrease and down regulate the intensity of the inflammatory response and restore normal nasal and sinus function.

As used herein, the terms “treat,” “treatment,” “treating,” and grammatical equivalents thereof refer to any manner in which one or more of the symptoms of the aforementioned conditions, disorders, or diseases are beneficially altered. Current treatments for relief of sinusitis, rhinitis, nasopharyngitis, nasal congestion, and any other issues related to the nasal airway and sinuses may include utilizing a nasal rinse. The term “nasal rinse,” as used herein, refers to any type of composition and method of the present disclosure that is used for achieving a desired function and/or for a desired purpose. Various nasal rinse delivery systems have been deployed, including bag-on-valve systems, ceramic neti pots, plastic bottles, squeeze bottles, and bulb syringes. As such, a novel nasal rinse deployed with a delivery system may decrease inflammation, reduce mucus viscosity, and promote mucociliary clearance, which may provide relief or permanently cure the symptoms associated with sinus and nasal disorders.

### DESCRIPTION OF CERTAIN EMBODIMENTS

The present disclosure relates to compositions and methods for treating one or more nasal passages. More particularly, the present disclosure relates to compositions and methods for a nasal rinse treatment fluid that is used for treating one or more nasal passages.

The present disclosure provides a composition including N-acetylcysteine, a salt, and an alkaline buffering agent. The present disclosure also provides methods that include adding the composition to an aqueous fluid to form a nasal rinse treatment fluid. In some embodiments, the methods may further include adding one or more essential oils to a nasal rinse treatment fluid. In some embodiments, the methods may further include adding one or more preservatives to a nasal rinse treatment fluid. In some embodiments, the methods may further include allowing the composition to increase the pH of a nasal rinse treatment fluid. In some embodiments, the methods may further include allowing the composition to increase the salt concentration of the nasal rinse treatment fluid to change from an isotonic solution to a hypertonic solution.

Among the many potential advantages to the methods and compositions of the present disclosure, only some of which are alluded to herein, the methods and compositions of the present disclosure may provide for reduced inflammation of one or more nasal passages. For example, in some embodiments, the methods and compositions of the present disclosure may reduce nasal infection, rhinitis, or sinusitis when the nasal rinse treatment fluid is administered in one or more nasal passages. Accordingly, the methods and compositions of the present disclosure may reduce facial pressure and pain that results from the impairment of sinus function and drainage. Moreover, in some embodiments, the methods and compositions of the present disclosure may provide a nasal rinse treatment fluid that is a mucolytic agent that will reduce mucus viscosity and promote mucociliary clearance in one or more nasal passages. Furthermore, the methods and compositions of the present disclosure may enhance respiratory functions, particularly as compared with the respiratory conditions associated with allergic rhinitis, acute rhinitis, chronic rhinitis, allergic sinusitis, acute sinusitis, chronic sinusitis, cystic fibrosis, and any other conditions marked by excess mucus.

In certain embodiments, the composition of the present disclosure may include N-acetylcysteine, a salt, and an alkaline buffering agent. In some embodiments, the composition of the present disclosure may include N-acetylcysteine, a salt, and an alkaline buffering agent in the form of a crystallized powder. In other embodiments, the composition of the present disclosure may include N-acetylcysteine, a salt, and an alkaline buffering agent in the form of an aqueous solution. In yet other embodiments, the composition of the present disclosure may include N-acetylcysteine, a salt, an alkaline buffering agent, an essential oil, and a preservative in the form of an aqueous solution.

The term “N-acetylcysteine,” as used herein, refers to an acetylated cysteine residue wherein an acetyl group is attached to the nitrogen atom. Without intending to be limited to any particular theory or mechanism, it is believed that, in certain embodiments, N-acetylcysteine is a mucolytic agent that moderates mucous secretions. The mucolytic action is due to N-acetylcysteine breaking the disulphide bridges in the glycoproteins of mucus, resulting in reduced viscosity. Additionally, N-acetylcysteine is believed to demonstrate antimicrobial, antioxidant, and antiviral properties. For instance, N-acetylcysteine may inhibit replication of the seasonal human influenza virus. As such, a nasal rinse treatment fluid including N-acetylcysteine may reduce inflammation of the mucosa and allow the sinuses to drain normally, direct antimicrobial activity or stimulate increased host antimicrobial activity, as well as provide an additional treatment option against human influenza.

In certain embodiments, N-acetylcysteine may be present in a composition of the present disclosure in an amount sufficient to reduce mucus viscosity and promote mucociliary clearance in one or more nasal passages. For instance, in some embodiments, the composition may include N-acetylcysteine in an amount sufficient to reduce inflammation in one or more nasal passages. In other embodiments, the composition may include N-acetylcysteine in an amount sufficient to reduce nasal infection, rhinitis, or sinusitis when the composition is administered in one or more nasal passages.

In certain embodiments, the composition of the present disclosure including N-acetylcysteine may be added to a crystallized powder. In certain embodiments, the composition may include N-acetylcysteine in an amount from about 1 to about 70 weight percent (“wt %”) by weight of the crystallized powder. In some embodiments, the composition may include N-acetylcysteine in an amount from about 5 wt % to about 50 wt % by weight of the crystallized powder. In other embodiments, the composition may include N-acetylcysteine in an amount from about 10 wt % to about 30 wt % by weight of the crystallized powder. In yet other embodiments, the composition may include N-acetylcysteine in an amount about 20 wt % by weight of the crystallized powder.

In certain embodiments, the composition of the present disclosure including N-acetylcysteine may be added to a nasal rinse treatment fluid, or to an aqueous fluid to form a nasal rinse treatment fluid. In certain embodiments, the composition may include N-acetylcysteine in an amount from about 0.0125 to about 10 volume percent (“vol %”) by volume of the nasal rinse treatment fluid. In some embodiments, the composition may include N-acetylcysteine in an amount from about 0.125 vol % to about 1 vol % by volume of the nasal rinse treatment fluid. In other embodiments, the composition may include N-acetylcysteine in an amount from about 0.25 vol % to about 0.5 vol % by volume of the nasal rinse treatment fluid.

In certain embodiments, one or more salts may be present in a composition of the present disclosure. Examples of salts suitable for certain embodiments of the present disclosure include, but are not limited to, sodium chloride, calcium chloride, ammonium chloride, magnesium chloride, strontium chloride, barium chloride, potassium chloride, ammonium sulfate, sodium sulfate, magnesium sulfate, aluminum sulfate, sodium bromide, potassium bromide, calcium bromide, zinc bromide, sodium bicarbonate, a carbonate salt, a sulfate salt, a phosphate salt, a magnesium salt, a bromide salt, a formate salt, an acetate salt, a chloride salt, a fluoride salt, a bicarbonate salt, a nitrate salt, a phosphate salt, and any combination thereof. In most embodiments, the composition of the present disclosure may include sodium chloride as the salt. The selection of a suitable salt may depend on several factors that would be recognized by a person of skill in the art with the benefit of this disclosure, including but not limited to any other types of salt in the composition of the present disclosure, the characteristics of an aqueous fluid (e.g., pH, salinity, etc.), and the like.

In certain embodiments, the composition of the present disclosure including a salt may be added to a nasal rinse treatment fluid, or to an aqueous fluid to form a nasal rinse treatment fluid. In certain embodiments, the salt may be present in a composition of the present disclosure in an amount sufficient to provide an isotonic nasal rinse treatment fluid. In such embodiments, the isotonic nasal rinse treatment fluid may be compatible with the nasal and sinus mucosa to prevent burning or stinging during administration of the nasal rinse treatment fluid in one or more nasal passages. In contrast, in other embodiments, the salt may be present in a composition of the present disclosure in an increased amount to provide a hypertonic nasal rinse treatment fluid. In such embodiments, the composition may include the salt in an amount greater than 0.9 vol % by volume of the nasal rinse treatment fluid to provide a hypertonic nasal rinse treatment fluid. In some embodiments, the salt may be present in the nasal rinse treatment fluid of the present disclosure in an amount sufficient to soften and dislodge mucus lining the nasal passage, resulting in cleaning the nasal mucosa. In other embodiments, the salt may be present in a composition of the present disclosure in an amount sufficient to remove inflammatory mediators, such as prostaglandins and leukotrienes. In certain embodiments, the composition may include the salt in an amount from about 0.01 vol % to about 1.8 vol % by volume of the nasal rinse treatment fluid. In some embodiments, the composition may include the salt in an amount from about 0.45 vol % to about 1.8 vol % by volume of the nasal rinse treatment fluid. In other embodiments, the composition may include the salt in an amount from about 0.9 vol % to about 1.8 vol % by volume of the nasal rinse treatment fluid.

In certain embodiments, the composition of the present disclosure including a salt may be added to a crystallized powder. Any of the foregoing reasons to add a salt to a nasal rinse treatment fluid, or to an aqueous fluid to form a nasal rinse treatment fluid may be applicable to adding a salt to a crystallized powder. In certain embodiments, the composition may include a salt in an amount from about 1 wt % to about 90 wt % by weight of the crystallized powder. In some embodiments, the composition may include a salt in an amount from about 30 wt % to about 80 wt % by weight of the crystallized powder. In other embodiments, the composition may include a salt in an amount from about 50 wt % to about 70 wt % by weight of the crystallized powder. In yet other embodiments, the composition may include a salt in an amount about 62.4 wt % by weight of the crystallized powder.

In certain embodiments, the composition of the present disclosure including an alkaline buffering agent may be added to a nasal rinse treatment fluid, or to an aqueous fluid to form a nasal rinse treatment fluid. In certain embodiments, the composition of the present disclosure may include an alkaline buffering agent that maintains the pH of the nasal rinse treatment fluid in a range of about 6 to about 9. In some embodiments, the alkaline buffering agent maintains the pH of the nasal rinse treatment fluid in a range of about 7 to about 8. In other embodiments, the alkaline buffering agent maintains the pH of the nasal rinse treatment fluid in a range of about 7.3 to about 7.5. In yet other embodiments, the alkaline buffering agent maintains the pH of the nasal rinse treatment fluid at about 7.4. Examples of alkaline buffering agents suitable for certain embodiments of the present disclosure include, but are not limited to, bicarbonate salts, carbonate salts, sodium or potassium hydroxides, high pH chelating agents, high pH surfactants, and any combination thereof. In most embodiments, the composition of the present disclosure may include sodium bicarbonate as the alkaline buffering agent. A person of skill in the art, with the benefit of this disclosure, would be able to a select an alkaline buffering agent that may be suitable for a particular application of the present disclosure.

The alkaline buffering agent may be present in a composition of the present disclosure in an amount sufficient to maintain a desired pH when the nasal rinse treatment fluid is administered in one or more nasal passages. For example, in certain embodiments, the alkaline buffering agent may be present in a composition of the present disclosure in an amount from about 0.01 vol % to about 1 vol % by volume of the nasal rinse treatment fluid. In some embodiments, the alkaline buffering agent may be present in a composition of the present disclosure in an amount from about 0.1 vol % to about 0.52 vol % by volume of the nasal rinse treatment fluid. In other embodiments, the alkaline buffering agent may be present in a composition of the present disclosure in an amount from about 0.2 vol % to about 0.3 vol % by volume of the nasal rinse treatment fluid.

In certain embodiments, the composition of the present disclosure including an alkaline buffering agent may be added to a crystallized powder. Any of the foregoing reasons to add an alkaline buffering agent to a nasal rinse treatment fluid, or to an aqueous fluid to form a nasal rinse treatment fluid may be applicable to adding an alkaline buffering agent to a crystallized powder. In certain embodiments, the composition may include an alkaline buffering agent in an amount from about 1 wt % to about 60 wt % by weight of the crystallized powder. In some embodiments, the composition may include an alkaline buffering agent in an amount from about 5 wt % to about 40 wt % by weight of the crystallized powder. In other embodiments, the composition may include an alkaline buffering agent in an amount from about 10 wt % to about 25 wt % by weight of the crystallized powder. In yet other embodiments, the composition may include an alkaline buffering agent in an amount about 17.6 wt % by weight of the crystallized powder.

In certain embodiments, the methods of the present disclosure may include adding a composition including N-acetylcysteine, a salt, and an alkaline buffering agent to an aqueous fluid. The aqueous fluid may include any aqueous fluid known in the art. Aqueous fluids that may be suitable for use in the methods and compositions of the present disclosure may include water from any source. Such aqueous fluids may include fresh water, salt water (e.g., water containing one or more salts dissolved therein), brine (e.g., saturated salt water), seawater, produced water, surface water (e.g., from a river or a pond), reclaimed water, and any combination thereof. In most embodiments of the present disclosure, the preferred aqueous fluids meet the standard of the United States Pharmacopeia (“USP”) grade for purified water. The USP grade purified water may be made using any suitable method, for example, reverse osmosis, carbon filtering, deionization, and any combination thereof. In other embodiments, the aqueous fluids may undergo any type of water filtration process to make the aqueous fluids suitable to add to a composition of the present disclosure.

In certain embodiments, the methods of the present disclosure may include adding a composition including N-acetylcysteine, a salt, and an alkaline buffering agent to a nasal rinse treatment fluid, or to an aqueous fluid to form a nasal rinse treatment fluid. In certain embodiments, the composition is present in the nasal rinse treatment fluid in an amount sufficient to reduce nasal infection, rhinitis, or sinusitis when the nasal rinse treatment fluid is administered in one or more nasal passages. For example, in some embodiments, the composition may be added to the nasal rinse treatment fluid in an amount from about 0.01 vol % to about 30 vol % by volume of the nasal rinse treatment fluid. In some embodiments, the composition may be added to the nasal rinse treatment fluid in an amount from about 0.1 vol % to about 5 vol % by volume of the nasal rinse treatment fluid. In other embodiments, the composition may be added to the nasal rinse treatment fluid in an amount from about 1 vol % to about 3 vol % by volume of the nasal rinse treatment fluid. Those of ordinary skill in the art will appreciate that the composition added to a nasal rinse treatment fluid should have a vol % sufficient in order to achieve a desired function.

In certain embodiments, the methods of the present disclosure may include adding one or more essential oils to the nasal rinse treatment fluid including a composition of the present disclosure. In certain embodiments, the one or more essential oils may be added to the nasal rinse treatment fluid after the composition has been added to the nasal rinse treatment fluid. Without intending to be limited to any particular theory or mechanism, it is believed that, in certain embodiments, essential oils may have antimicrobial activity against a wide range of pathogens, with various spectrums of activity. The antimicrobial action is believed to result from the composition and cytotoxic effects of essential oils, which cause cell membrane damage. As such, a nasal rinse treatment fluid including essential oils may enhance the antimicrobial activity of the nasal rinse treatment fluid. For instance, in some embodiments, the essential oils may help reduce gram-positive and gram-negative bacteria growth. Examples of such essential oils for certain embodiments of the present disclosure include, but are not limited to, tea tree oil, eucalyptus oil, peppermint oil, carrot seed oil, cinnamon oil, rosemary oil, oregano oil, thyme oil, any other essential oils that help reduce gram-positive and gram-negative bacteria growth, and any combination thereof. In certain embodiments, the one or more essential oils may be added to the nasal rinse treatment fluid in an amount from about 0.0125 vol % to about 10 vol % by volume of the nasal rinse treatment fluid. In some embodiments, the one or more essential oils may be added to the nasal rinse treatment fluid in an amount from about 0.125 vol % to about 1 vol % by volume of the nasal rinse treatment fluid. In other embodiments the one or more essential oils may be added to the nasal rinse treatment fluid in an amount from about 0.1 vol % to about 0.3 vol % by volume of the nasal rinse treatment fluid.

In certain embodiments, the methods of the present disclosure may include adding one or more preservatives to the nasal rinse treatment fluid including a composition of the present disclosure. In certain embodiments, the one or more preservatives may be added to the nasal rinse treatment fluid after the composition has been added to the nasal rinse treatment fluid. In some embodiments, the preservatives may help reduce the growth of microorganisms that may be introduced into the nasal rinse treatment fluid after opening its storage container. Examples of such preservatives for certain embodiments of the present disclosure include, but are not limited to, grapefruit seed extract, benzalkonium chloride, benzyl alcohol, sodium benzyl chloride, sodium benzoate, potassium sorbate, potassium benzoate, sodium benzyl alcohol, any other preservatives that help reduce the growth of microorganisms, and any combination thereof. In certain embodiments, the one or more preservatives may be added to the nasal rinse treatment fluid in an amount from about 0.0125 vol % to about 10 vol % by volume of the nasal rinse treatment fluid. In some embodiments, the one or more preservatives may be added to the nasal rinse treatment fluid in an amount from about 0.125 vol % to about 1 vol % by volume of the nasal rinse treatment fluid. In other embodiments the one or more preservatives may be added to the nasal rinse treatment fluid in an amount from about 0.25 vol % to about 0.5 vol % by volume of the nasal rinse treatment fluid.

In certain embodiments, the components of the present disclosure including N-acetylcysteine, a salt, and an alkaline buffering agent may be combined together before being added to a nasal rinse treatment fluid, or to an aqueous fluid to form a nasal rinse treatment fluid. In such embodiments, compositions including one or more of those components may be stored (e.g., in a sachet or a container) for a period of time before being added to the nasal rinse treatment fluid. In certain embodiments, such compositions of the present disclosure may be a crystallized powder. In certain embodiments, such compositions may be stored for up to 48 months before being added to a nasal rinse treatment fluid, or to an aqueous fluid to form a nasal rinse treatment fluid. In some embodiments, such compositions may be stored for up to 6 months before being added to a nasal rinse treatment fluid, or to an aqueous fluid to form a nasal rinse treatment fluid. After the composition is added to the nasal rinse treatment fluid, in some embodiments, the one or more essential oils and one or more preservatives may be added to the nasal rinse treatment fluid.

In other embodiments, the components of the present disclosure including N-acetylcysteine, a salt, an alkaline buffering agent, an essential oil, and a preservative may be added separately to a nasal rinse treatment fluid, or to an aqueous fluid to form a nasal rinse treatment fluid. In such embodiments, N-acetylcysteine, a salt, an alkaline buffering agent, an essential oil, and a preservative may collectively constitute a composition even when separately added to the nasal rinse treatment fluid. In certain embodiments, the composition (or one or more components thereof) may be added to the nasal rinse treatment fluid by batch mixing or continuous mixing. Examples of mixing devices suitable for certain embodiments of the present disclosure include, but are not limited to, a ribbon agitator, a paddle agitator, a rotary shell agitator, a double-cone agitator, a hybrid agitator, any agitator that helps mix components of the present disclosure, and any combination thereof. In other embodiments, the composition (or one or more components thereof) may be directly added to a tank or other suitable vessel containing the nasal rinse treatment fluid.

In one or more embodiments, the nasal rinse treatment fluid of the present disclosure may be administered into one or more nasal passages using a bag-on-valve delivery system. A user of the bag-on-valve delivery system may rinse one or more nasal passages to reduce an inflammatory response and restore normal nasal and sinus function. In certain embodiments, the bag-on-valve delivery system may provide a continuous distribution of the nasal rinse treatment fluid at all angles in one or more nasal passages. In such embodiments, the nasal rinse treatment fluid may be placed inside a storage container of the bag-on-valve delivery system, which uses a propellant in conjunction to dispense the nasal rinse treatment fluid. In administering the nasal rinse treatment fluid of the present disclosure into one or more nasal passages, the components of the nasal rinse treatment fluid may be mixed together in the storage container of the bag-on-valve delivery system, or one or more components may mix or intermingle upon dispensing the nasal rinse treatment fluid from the bag-on-valve delivery system. In either such case, the nasal rinse treatment fluid is deemed to be introduced into one or more nasal passages for purposes of the present disclosure.

In one or more embodiments, the composition of the present disclosure may be a crystallized powdered that is stored in an individually packaged sachet. In such embodiments, the crystallized powder that is stored in a sachet may be added to an aqueous fluid to form a nasal rinse treatment fluid. The individually packaged sachets may be sized for a single nasal rinse treatment fluid application or multiple nasal rinse treatment fluid applications. For example, in certain embodiments, the crystallized powder may be added to an aqueous fluid, in which the amount of aqueous fluid is from about 1 milliliter (“mL”) to about 10,000 mL. In some embodiments, the crystallized powder may be added to an aqueous fluid, in which the amount of aqueous fluid is from about 100 mL to about 1,000 mL. In other embodiments, the crystallized powder may be added to an aqueous fluid, in which the amount of aqueous fluid is from about 150 mL to about 350 mL. In yet other embodiments, the crystallized powder may be added to an aqueous fluid, in which the amount of aqueous fluid is about 300 mL. The resultant nasal rinse treatment fluid may be used for nasal irrigation, in which one or more nasal passages are washed with the nasal rinse treatment fluid for purposes of the present disclosure.

An embodiment of the present disclosure is a composition that includes: N-acetylcysteine, a salt, and an alkaline buffering agent.

In one or more embodiments described in the preceding paragraph, the salt includes sodium chloride. In one or more embodiments described in the preceding paragraph, the alkaline buffering agent includes sodium bicarbonate. In one or more embodiments described in the preceding paragraph, the composition is formed into a crystallized powder. In one or more embodiments described in the preceding paragraph, the N-acetylcysteine is present in the composition in an amount from about 1 wt % to about 70 wt % by weight of the crystallized powder. In one or more embodiments described in the preceding paragraph, the salt is present in the composition in an amount from about 1 wt % to about 90 wt % by weight of the crystallized powder. In one or more embodiments described in the preceding paragraph, the alkaline buffering agent is present in the composition in an amount from about 1 wt % to about 60 wt % by weight of the crystallized powder. In one or more embodiments described in the preceding paragraph, the composition is stored in an individually packaged sachet. In one or more embodiments described in the preceding paragraph, the composition further includes an aqueous fluid. In one or more embodiments described in the preceding paragraph, the composition further includes an essential oil and a preservative.

An embodiment of the present disclosure is a method that includes: providing a composition that includes N-acetylcysteine, a salt, and an alkaline buffering agent; introducing the composition into an aqueous fluid to form a nasal rinse treatment fluid; and introducing the nasal rinse treatment fluid into one or more nasal passages.

In one or more embodiments described in the preceding paragraph, the N-acetylcysteine is present in the nasal rinse treatment fluid in an amount from about 0.0125 vol % to about 10 vol % by volume of the nasal rinse treatment fluid. In one or more embodiments described in the preceding paragraph, the salt includes at least one salt selected from the group consisting of a chloride salt, a carbonate salt, a sulfate salt, a phosphate salt, a magnesium salt, a bromide salt, a formate salt, an acetate salt, a fluoride salt, a bicarbonate salt, a nitrate salt, a phosphate salt, and any combination thereof. In one or more embodiments described in the preceding paragraph, the salt is present in the nasal rinse treatment fluid in an amount from about 0.01 vol % to about 1.8 vol % by volume of the nasal rinse treatment fluid. In one or more embodiments described in the preceding paragraph, the alkaline buffering agent includes at least one alkaline buffering agent selected from the group consisting of a bicarbonate salt, a carbonate salt, a hydroxide salt, a high pH chelating agent, a high pH surfactant, and any combination thereof. In one or more embodiments described in the preceding paragraph, the alkaline buffering agent is present in the nasal rinse treatment fluid in an amount from about 0.01 vol % to about 1 vol % by volume of the nasal rinse treatment fluid. In one or more embodiments described in the preceding paragraph, the alkaline buffering agent is present in the composition in an amount sufficient to increase the pH of the nasal rinse treatment fluid to a level between about 6 and about 9. In one or more embodiments described in the preceding paragraph, the nasal rinse treatment fluid further includes at least one essential oil selected from the group consisting of a tea tree oil, eucalyptus oil, peppermint oil, carrot seed oil, cinnamon oil, rosemary oil, oregano oil, thyme oil, and any combination thereof. In one or more embodiments described in the preceding paragraph, the nasal rinse treatment fluid further includes at least one preservative selected from the group consisting of grapefruit seed extract, benzalkonium chloride, benzyl alcohol, sodium benzyl chloride, sodium benzoate, potassium sorbate, potassium benzoate, sodium benzyl alcohol, and any combination thereof. In one or more embodiments described in the preceding paragraph, further including introducing the nasal rinse treatment fluid into one or more nasal passages using a bag-on-valve system.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. While numerous changes may be made by those skilled in the art, such changes are encompassed within the spirit of the subject matter defined by the appended claims. Furthermore, no limitations are intended to the details of construction or design herein shown, other than as described in the claims below. It is therefore evident that the particular illustrative embodiments disclosed above may be altered or modified and all such variations are considered within the scope and spirit of the present disclosure. In particular, every range of values (e.g., “from about a to about b,” or, equivalently, “from approximately a to b,” or, equivalently, “from approximately a-b”) disclosed herein is to be understood as referring to the power set (the set of all subsets) of the respective range of values. The terms in the claims have their plain, ordinary meaning unless otherwise explicitly and clearly defined by the patentee.

What is claimed is:

1. A composition comprising:
N-acetylcysteine,
a salt; and
an alkaline buffering agent.

2. The composition of claim 1, wherein the salt comprises sodium chloride.

3. The composition of claim 1, wherein the alkaline buffering agent comprises sodium bicarbonate.

4. The composition of claim 1, wherein the composition is formed into a crystallized powder.

5. The composition of claim 4, wherein the N-acetylcysteine is present in the composition in an amount from about 1 wt % to about 70 wt % by weight of the crystallized powder.

6. The composition of claim 4, wherein the salt is present in the composition in an amount from about 1 wt % to about 90 wt % by weight of the crystallized powder.

7. The composition of claim 4, wherein the alkaline buffering agent is present in the composition in an amount from about 1 wt % to about 60 wt % by weight of the crystallized powder.

8. The composition of claim 1, wherein the composition is stored in an individually packaged sachet.

9. The composition of claim 1, wherein the composition further comprises an aqueous fluid.

10. The composition of claim 9, wherein the composition further comprises an essential oil and a preservative.

11. A method comprising:
providing a composition that comprises:
N-acetylcysteine;
a salt; and
an alkaline buffering agent;
introducing the composition into an aqueous fluid to form a nasal rinse treatment fluid; and
introducing the nasal rinse treatment fluid into one or more nasal passages.

12. The method of claim 11, wherein the N-acetylcysteine is present in the nasal rinse treatment fluid in an amount from about 0.0125 vol % to about 10 vol % by volume of the nasal rinse treatment fluid.

13. The method of claim 11, wherein the salt comprises at least one salt selected from the group consisting of a chloride salt, a carbonate salt, a sulfate salt, a phosphate salt, a magnesium salt, a bromide salt, a formate salt, an acetate salt, a fluoride salt, a bicarbonate salt, a nitrate salt, a phosphate salt, and any combination thereof.

14. The method of claim 11, wherein the salt is present in the nasal rinse treatment fluid in an amount from about 0.01 vol % to about 1.8 vol % by volume of the nasal rinse treatment fluid.

15. The method of claim 11, wherein the alkaline buffering agent comprises at least one alkaline buffering agent selected from the group consisting of a bicarbonate salt, a carbonate salt, a hydroxide salt, a high pH chelating agent, a high pH surfactant, and any combination thereof.

16. The method of claim 11, wherein the alkaline buffering agent is present in the nasal rinse treatment fluid in an amount from about 0.01 vol % to about 1 vol % by volume of the nasal rinse treatment fluid.

17. The method of claim 11, wherein the alkaline buffering agent is present in the composition in an amount sufficient to increase the pH of the nasal rinse treatment fluid to a level between about 6 and about 9.

18. The method of claim 11, wherein the nasal rinse treatment fluid further comprises at least one essential oil selected from the group consisting of a tea tree oil, eucalyptus oil, peppermint oil, carrot seed oil, cinnamon oil, rosemary oil, oregano oil, thyme oil, and any combination thereof.

19. The method of claim 11, wherein the nasal rinse treatment fluid further comprises at least one preservative selected from the group consisting of grapefruit seed extract, benzalkonium chloride, benzyl alcohol, sodium benzyl chloride, sodium benzoate, potassium sorbate, potassium benzoate, sodium benzyl alcohol, and any combination thereof.

20. The method of claim 11, further comprising introducing the nasal rinse treatment fluid into one or more nasal passages using a bag-on-valve system.
